(19) United States
(12) Patent Application Publication
(10) Pub. No.: US 2011/0028746 A1
(43) Pub. Date: Feb. 3, 2011

(54) METHOD FOR THE DECARBOXYLATIVE HYDROFORMYLATION OF ALPHA, BETA- UNSATURATED CARBOXYLIC ACIDS

(75) Inventors: Jens Rudolph, Worms (DE); Joachim Schmidt-Leithoff, Mannheim (DE); Rocco Paciello, Bad Durkheim (DE); Bernhard Breit, Gundelfingen (DE); Thomas Smejkal, Freiburg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/934,743

(22) PCT Filed: Mar. 25, 2009
(86) PCT No.: PCT/EP2009/053523

§ 371 Date: Sep. 27, 2010

(30) Foreign Application Priority Data
Mar. 26, 2008 (DE) 102008015773.2

(51) Int. Cl. C07C 45/49; C07C 319/12; C07F 7/08; C07C 51/14; C07C 269/06; C07C 67/36

(52) U.S. Cl. 554/115; 568/454; 568/444; 568/429; 568/41; 556/436; 568/387; 560/24; 560/51

(57) ABSTRACT

The present invention relates to a process for preparing aldehydes by reacting an α,β-unsaturated carboxylic acid or a salt thereof with carbon monoxide and hydrogen in the presence of a catalyst comprising at least one complex of a metal of transition group VIII of the Periodic Table of the Elements with at least one compound of the formula (I),

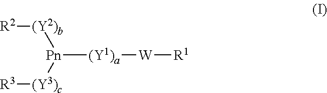

where Pn is pnicogen; W is a divalent bridging group having from 1 to 8 bridge atoms between the flanking bonds; R¹ is a functional group capable of forming at least one intermolecular, noncovalent bond with the —X(═O)OH group of the compound of the formula (I); R², R³ are each in each case optionally substituted alkyl, cycloalkyl, heterocycloalkyl, aryl or hetaryl or together with the pnicogen atom and together with the groups Y² and Y³ if present form an optionally fused and optionally substituted 5- to 8-membered heterocycle; a, b and c are each 0 or 1; and Y^{1,2,3} are each, independently of one another, O, S, NR^{a} or SiR^{b}R^{c}, where R^{a,b,c} are each H or in each case optionally substituted alkyl, cycloalkyl, heterocycloalkyl, aryl or hetaryl; and the use of the above-described catalyst for the decarboxylative hydroformylation of α,β-unsaturated carboxylic acids.

The present invention relates to a process for preparing aldehydes by reaction of an α,β-unsaturated carboxylic acid or a salt thereof with carbon monoxide and hydrogen in the presence of a catalyst comprising a complex of a metal of transition group VIII with a pnicogen-comprising compound as ligand, where the pnicogen-comprising compound has a functional group which is complementary to the carboxyl group of the α,β-unsaturated carboxylic acid to be reacted, and also the use of such catalysts for the decarboxylative hydroformylation of α,β-unsaturated carboxylic acids.

The use of ligands capable of dimerization in hydroformylation catalysts, i.e. ligands capable of forming aggregates, is described, for example, in B. Breit and W. Seiche, J. Am. Chem. Soc. 2003, 125, 6608-6609, in EP 1 486 481, in PCT/EP 2007/059722 or in DE 10 2006 041 064. However, none of the abovementioned documents describes the ability of the ligands to aggregate with the compound to be reacted (substrate).

In Angew. Chem. 2008, 120, 2, 317-321, B. Breit and T. Smejkal describe the hydroformylation of unsaturated carboxylic acids in the presence of ligands of the formulae (1) and (2)

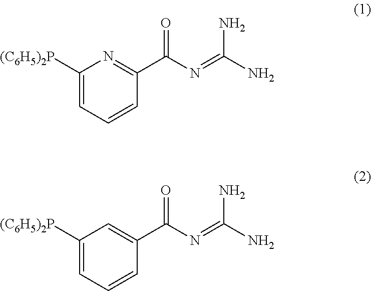

These ligands are capable of interacting with the carboxyl group of the unsaturated carboxylic acids to be hydroformylated. In this way, a high regioselectivity and chemoselectivity of the hydroformylation reaction in respect of the functional group reacted is achieved. However, the reaction of α,β-unsaturated carboxylic acids in the presence of the catalysts described under hydroformylation conditions is not described in this article.

It is an object of the present invention to provide a process which is suitable for the chemoselective conversion of α,β-unsaturated carboxylic acids into the corresponding α,β-saturated aldehydes. The process should be suitable for hydrogenating the conjugated C—C double bond of the α,β-unsaturated carboxylic acid in high yield and at the same time converting the carboxylic acid group into an aldehyde group with high selectivity and in high yield. Furthermore, it should be possible to employ the process in the presence of further functional groups such as double bonds, functional groups comprising carbonyl groups or hydrolysis-sensitive protective groups with high selectivity over undesirable secondary reactions. In particular, in the reaction of α,β-unsaturated carboxylic acids comprising further, nonconjugated double bonds, the process should not result in any hydrogenation an/or isomerization of the further double bonds.

It has now surprisingly been found that this object is achieved by the reaction of α,β-unsaturated carboxylic acids with carbon monoxide and hydrogen in the presence of catalysts whose ligands are capable of forming intermolecular, noncovalent bonds with the carboxyl group of the α,β-unsaturated carboxylic acids to be reacted (substrate). This reaction will hereinafter be referred to as decarboxylative hydroformylation. As a result of the “substrate recognition”, a high selectivity in respect of the substrate reacted or the functional group reacted is achieved. The process of the invention is therefore also suitable, in particular, for the selective hydrogenation of the conjugated double bond and replacement of the carboxyl group by an aldehyde group on α,β-unsaturated carboxylic acids which have further functional groups capable of reacting under customary reduction conditions.

The present invention therefore provides a process for preparing aldehydes by reacting an α,β-unsaturated carboxylic acid or a salt thereof with carbon monoxide and hydrogen in the presence of a catalyst

comprising at least one complex of a metal of transition group VIII of the Periodic Table of the Elements with at least one compound of the formula (I),

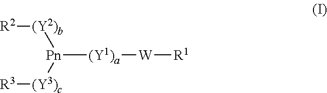

where
Pn is a pnicogen atom,
W is a divalent bridging group having from 1 to 8 bridge atoms between the flanking bonds,
R¹ is a functional group capable of forming at least one intermolecular, noncovalent bond with the —X(═O)OH group of the compound of the formula (I),
R² and R³ are each, independently of one another, alkyl, cycloalkyl, heterocycloalkyl, aryl or hetaryl, where alkyl is unsubstituted or substituted by 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl are unsubstituted or substituted by 1, 2, 3, 4 or 5 substituents selected from among alkyl and the substituents mentioned above for alkyl; or together with the pnicogen atom and together with the groups Y² and Y³ if present form a 5- to 8-membered heterocycle which may additionally be fused with one, two, three or four cycloalkyl, heterocycloalkyl, aryl or hetaryl groups, where the heterocycle and, if present, the fused-on groups each have, independently of one another, 1, 2, 3, 4 or 5 substituents selected from among halogen, cyano, nitro, alkyl, alkoxy, cycloalkyl, cycloalkoxy, heterocycloalkyl, heterocycloalkoxy, aryl, aryloxy, hetaryl and hetaryloxy,
a, b and c are each, independently of one another, 0 or 1 and
Y¹, Y² and Y³ are each, independently of one another, O, S, NR^{a} or SiR^{b}R^{c}, where R^{a}, R^{b} and R^{c} are each, independently of one another, hydrogen, alkyl, cycloalkyl, heterocycloalkyl, aryl or hetaryl, where alkyl is unsubstituted or substituted by 1, 2, 3, 4 or 5 substituents selected from among halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl are unsubstituted or substituted by 1, 2, 3, 4 or 5 substituents selected from among alkyl and the substituents mentioned above for alkyl.

The process of the invention is distinguished, in particular, by the fact that the number of carbon atoms of the aldehyde produced corresponds to the number of carbon atoms of the α,β-unsaturated carboxylic acid used.

According to the invention, ligands of the formula (I) which have a functional group R¹ capable of forming intermolecular, noncovalent bonds with the carboxyl group of the α,β-unsaturated carboxylic acid are used. These bonds are preferably hydrogen bonds or ionic bonds, in particular hydrogen bonds. The functional groups capable of forming intermolecular noncovalent bonds make the ligands capable of association with α,β-unsaturated carboxylic acids, i.e. of formation of aggregates in the form of heterodimers.

A pair of functional groups of the ligands and of the α,β-unsaturated carboxylic acid which are capable of forming intermolecular noncovalent bonds will for the purposes of the present invention be referred to as “complementary”. “Complementary compounds” are ligand/carboxylic acid pairs which have functional groups which are complementary to one another. Such pairs are capable of association, i.e. of forming aggregates.

For the purposes of the present invention, “halogen” is fluorine, chlorine, bromine or iodine, preferably fluorine, chlorine or bromine.

For the purposes of the present invention, “pnicogen” is phosphorus, arsenic, antimony and bismuth, in particular phosphorus.

For the purposes of the present invention, “alkyl” is a straight-chain or branched alkyl group. It is preferably a straight-chain or branched C₁-C₂₀-alkyl, preferably C₁-C₁₂-alkyl, particularly preferably C₁-C₈-alkyl and very particularly preferably C₁-C₄-alkyl group. Examples of alkyl groups are, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression “alkyl” also comprises substituted alkyl groups which generally have 1, 2, 3, 4 or 5 substituents, preferably 1, 2 or 3 substituents and particularly preferably 1 substituent. These are preferably selected from among halogen, cyano, nitro, alkoxy, cycloalkyl, cycloalkoxy, heterocycloalkyl, heterocycloalkoxy, aryl, aryloxy, hetaryl and hetaryloxy.

The expression “alkyl” also comprises alkyl groups in which one or more, in particular from 1 to 5, nonadjacent CH₂ groups are, independently of one another, replaced by —O—, —O—C(═O)—, —O—Si(R^{4a})(R^{4b})—, —O—C(═O)—O—, —O—C(═O)—N(R^{4c})—, —O—C(═O)—S—, —N(R^{4c})—, —N(R^{4c})—C(═O)—,—N(R^{4c})—C(═O)—O—, —N(R^{4c})—C(═O)—N(R^{4c})—, —N(R^{4c})—C(═O)—S—, —S—, —S—C(═O)—, —S—C(═O)—O—, —S—C(═O)—N(R^{4c})—, —S—C(═O)—S—, —C(═O)—, —C(═O)—O—, —C(═O)—N(R^{4c})—, —C(═O)—S— or —Si(R^{4b})(R^{4c})—where R^{4a} and R^{4b} are each, independently of one another, alkyl and R^{4c} is H, alkyl, cycloalkyl, heterocycloalkyl, aryl or hetaryl. The CH₂ groups replaced can be either internal methylene groups of the alkyl groups or the methylene part of a terminal methyl group. Examples of such alkyl groups are accordingly in each case unsubstituted or substituted hydroxyalkyl, alkoxyalkyl, hydroxycarbonylalkyl, alkoxycarbonylalkyl, trialkylsilyloxyalkyl, hydroxycarbonyloxyalkyl, alkoxycarbonyloxyalkyl, N-(hydroxycarbonyl)aminoalkyl, N-(hydroxycarbonyl)-N-alkylaminoalkyl, N-(alkoxycarbonyl)aminoalkyl, N-(alkoxycarbonyl)-N-alkylaminoalkyl, hydroxycarbonylsulfanylalkyl, alkoxycarbonylsulfanylalkyl, aminoalkyl, N-alkylaminoalkyl, N,N-dialkylaminoalkyl, aminocarbonylalkyl, N-alkylaminocarbonylalkyl, N,N-dialkylaminocarbonylalkyl, aminocarbonyloxyalkyl, N-alkylaminocarbonyloxyalkyl, N,N-dialkylaminocarbonyloxyalkyl, N-(aminocarbonyl)aminoalkyl, N-(N′-alkylaminocarbonyl)aminoalkyl, N-(N′,N′-dialkylaminocarbonyl)aminoalkyl, N-(aminocarbonyl)-, N-alkylaminoalkyl, N-(N′-alkylaminocarbonyl)-N-alkylaminoalkyl, N-(N′,N′-dialkylaminocarbonyl)-N-alkylaminoalkyl, aminocarbonylsulfanylalkyl, N-alkylaminocarbonylsulfanylalkyl, N,N-dialkylaminocarbonylsulfanylalkyl, thioalkyl, alkylsulfanylalkyl, alkylsulfanylcarbonylalkyl, alkylsulfanylcarbonyloxyalkyl, N-(alkylsulfanylcarbonyl)aminoalkyl, N-(alkylsulfanylcarbonyl)-N-alkylaminoalkyl, alkylsulfanylcarbonylsulfanylalkyl, formylalkyl, alkylcarbonylalkyl, alkylcarbonyloxyalkyl, N-(alkylcarbonyl)aminoalkyl, N-(alkylcarbonyl)-N-alkylaminoalkyl, alkylcarbonylsulfanylalkyl and trialkylsilylalkyl. It is likewise possible to use alkyl groups in which a plurality of CH₂ groups, for example from 2 to 5 CH₂ groups, have been replaced, i.e. alkyl groups which have a combination of two or more of the functional groups mentioned above by way of example. The abovementioned groups are in each case preferably derived from C₁-C₂₀-alkyl, particularly preferably from C₁-C₁₂-alkyl and very particularly preferably from C₁-C₈-alkyl.

If “alkyl” is an alkyl group in which one or more nonadjacent CH₂ groups have been replaced, the substituents, if present, are preferably selected from among halogen, cyano, nitro, cycloalkyl, heterocycloalkyl, aryl and hetaryl.

For the purposes of the present invention, the term “alkenyl” refers to unsubstituted or substituted, straight-chain or branched alkenyl groups having one or more double bonds. These are preferably straight-chain or branched C₂-C₂₀-alkenyl, preferably C₂-C₁₂-alkenyl, particularly preferably C₂-C₄-alkenyl and very particularly preferably C₂-C₄-alkenyl, groups. As regards suitable and preferred substituents, what has been said with regard to alkyl applies analogously.

The expression “alkenyl” also comprises alkenyl groups in which one or more, in particular from 1 to 5, nonadjacent CH₂ groups have, independently of one another, been replaced by —O—, —O—C(═O)—, —O—Si(R^{4a})(R^{4b})—, —O—C(═O)—O—, —O—C(═O)—N(R^{4c})—, —O—C(═O)—S—, —N(R^{4c})—, —N(R^{4c})—C(═O)—, —N(R^{4c})—C(═O)—O—, —N(R^{4c})—C(═O)—N(R^{4c}—, —N(R^{4c})—C(═O)—S—, —S—, —S—C(═O)—, —S—C(═O)—O—, —S—C(═O)—N(R^{4c})—, —S—C(═O)—S—, —C(═O)—, —C(═O)—O—, —C(═O)—N(R^{c}—, —C(═O)—S— or —Si(R^{4b})(R^{4c})— where R^{4a} and R^{4b} are each, independently of one another, alkyl and R^{4c} is H, alkyl, cycloalkyl, heterocycloalkyl, aryl or hetaryl. As regards suitable and preferred examples of these, what has been said with regard to alkyl applies analogously.

For the purposes of the present invention, the term “alkynyl” refers to unsubstituted or substituted, straight-chain or branched, monounsaturated or multiply unsaturated alkynyl groups. These are preferably straight-chain or branched C₂-C₂₀-alkynyl, preferably C₂-C₁₂-alkynyll and very particularly preferably C₂-C₄-alkynyl, groups. As regards suitable and preferred substituents, what has been said with regard to alkyl applies analogously.

The expression “alkynyl” also comprises alkynyl groups in which one or more, in particular from 1 to 5, nonadjacent CH₂ groups have, independently of one another, been replaced by —O—, —O—C(═O)—, —O—Si(R^{4a})(R^{4b})—, —O—C(═O)—O—, —O—C(═O)—N(R^{4c})—, —O—C(═O)—S—, —N(R^{4c})—, —N(R^{4c})—C(═O)—, —N(R^{4c})—C(═O)—O—, —N(R^{4c})—C(═O)—N(R^{4c})—, —N(R^{4c})—C(═O)—S—, —S—, —S—C(═O)—, —S—C(═O)—O—, —S—C(═O)—N(R^{4c})—, —S—C(═O)—S—, —C(═O)—, —C(═O)—O—, —C(═O)—N(R^{e})—, —C(═O)—S— or —Si(R^{4b})(R^{4c})—, where R^{4a} and R^{4b} are each, independently of one another, alkyl and R^{4c} is H, alkyl, cycloalkyl, heterocycloalkyl, aryl or hetaryl. As regards suitable and preferred examples of these, what has been said above with regard to alkyl applies analogously.

For the purposes of the present invention, the term “cycloalkyl” refers to unsubstituted or substituted cycloalkyl groups, preferably C₃-C₇-cycloalkyl groups such as cyclopropyl, cyclobutyl, cyclopentyl, cyclohexyl or cycloheptyl. These can, if they are substituted, generally bear 1, 2, 3, 4 or 5 substituents, preferably 1, 2 or 3 substituents and particularly preferably 1 substituent. These substituents are preferably selected from among alkyl, alkoxy and halogen.

For the purposes of the present invention, the term “heterocycloalkyl” refers to saturated, cycloaliphatic groups which generally have from 4 to 7, preferably 5 or 6, ring atoms and in which 1 or 2 of the ring carbons have been replaced by heteroatoms selected from among the elements O, N, S and P and are unsubstituted or substituted, in which case these heterocycloaliphatic groups can bear 1, 2 or 3 substituents, preferably 1 or 2 substituents, particularly preferably 1 substituent. These substituents are preferably selected from among alkyl, halogen, cyano, nitro, alkoxy, cycloalkyl, cycloalkoxy, heterocycloalkyl, heterocycloalkoxy, aryl, aryloxy, hetaryl and hetaryloxy, particularly preferably alkyl radicals. Examples of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

For the purposes of the present invention, the term “aryl” refers to unsubstituted or substituted aryl groups, preferably phenyl, tolyl, xylyl, mesityl, naphthyl, fluorenyl, anthracenyl, phenanthrenyl or naphthacenyl and particularly preferably phenyl or naphthyl, where these aryl groups can, if they are substituted, generally bear 1, 2, 3, 4 or 5 substituents, preferably 1, 2 or 3 substituents and particularly preferably one substituent, selected from among alkyl, halogen, cyano, nitro, alkoxy, cycloalkyl, cycloalkoxy, heterocycloalkyl, heterocycloalkoxy, aryl, aryloxy, hetaryl and hetaryloxy.

For the purposes of the present invention, the term “hetaryl” refers to unsubstituted or substituted, heterocycloaromatic groups which are preferably selected from among pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl. These heterocycloaromatic groups can, if they are substituted, generally bear 1, 2 or 3 substituents selected from among alkyl, halogen, cyano, nitro, alkoxy, cycloalkyl, cycloalkoxy, heterocycloalkyl, heterocycloalkoxy, aryl, aryloxy, hetaryl and hetaryloxy.

For the purposes of the present invention, the term “C₁-C₄-alkylene” refers to unsubstituted or substituted methylene, 1,2-ethylene, 1,3-propylene, 1,4-butylene, which, if it is substituted, can bear 1, 2, 3 or 4 substituents selected from among alkyl, halogen, cyano, nitro, alkoxy, cycloalkyl, cycloalkoxy, heterocycloalkyl, heterocycloalkoxy, aryl, aryloxy, hetaryl and hetaryloxy.

What has been said above with regard to the expressions “alkyl”, “cycloalkyl”, “heterocycloalkyl”, “aryl” and “hetaryl” applies analogously to the expressions “alkoxy”, “cycloalkoxy”, “heterocycloalkoxy”, “aryloxy” and “hetaryloxy”.

For the purposes of the present invention, “salts of the α,β-unsaturated carboxylic acids” are preferably alkali metal salts, in particular Na⁺, K⁺ and Li⁺ salts, alkaline earth metal salts, in particular Ca²⁺ or Mg²⁺ salts, or onium salts such as ammonium, monoalkylammonium, dialkylammonium, trialkylammonium, tetraalkylammonium, phosphonium, tetraalkylphosphonium or tetraarylphosphonium salts of the α,β-unsaturated carboxylic acids and in particular compounds of the formula M^{+ -}O—C(═O)—CH═CH—R⁴, where M₊ is a cation equivalent, i.e. a monovalent cation or the part of a polyvalent cation corresponding to a single positive charge. The cation M⁺ serves merely as counterion to the ⁻O—C(═O) group and can in principle be selected freely.

In the context of the present invention, the expression “decarboxylative hydroformylation” is used, without this implying a particular mechanism, to refer to reactions in which the conjugated C—C double bond of an α,β-unsaturated carboxylic acid is converted into a C—C single bond and the carboxylic group of the same α,β-unsaturated carboxylic acid is converted into an aldehyde group under hydroformylation conditions, i.e. on reaction with carbon monoxide and hydrogen in the presence of a hydroformylation catalyst. The reaction product of the decarboxylative hydroformylation is consequently an α,β-saturated aldehyde having the same number of carbon atoms as the α,β-unsaturated carboxylic acid which is reacted.

Without being tied to a theory, it is assumed that the catalyst comprising a metal of transition group VIII of the Periodic Table of the Elements and a compound of the formula (I), by means of the group R¹ capable of forming an intermolecular, noncovalent bond, forms an aggregate with the compound of the α,β-unsaturated carboxylic acids, with the C—C double bond of the α,β-unsaturated carboxylic acids being capable of interacting with the complexed metal of transition group VIII. Accordingly, a supramolecular, cyclic transition state could be transiently fowled here.

Compounds of the formula (I) in which Pn, R¹, R², R³, W, a, b, c, Y¹, Y², Y³ can independently or preferably in combination have one of the following meanings are particularly useful to the process of the invention.

Pn in the compounds of the formula (I) is preferably phosphorus. Suitable examples of such compounds of the formula (I) are phosphine, phosphinite, phosphonite, phosphoramidite or phosphite compounds.

R¹ in the compounds of the formula (I) is a functional group comprising at least one NH group. Suitable radicals R¹ are, for example, —NHR^{w}, ═NH, —C(═O)NHR^{w}, —C(═S)NHR^{w}, —C(═NR^{y})NHR^{w}, —O—C(═O)NHR^{w}, —O—C(═S)NHR^{w}, —O—C(═NR^{y})NHR^{w}, —N(R^{z})—C(═O)NHR^{w}, —N(R^{z})—C(═S)NHR^{w} or —N(R^{z})—C(═NR^{y})NHR^{w}, where R^{y} and R^{z} are each, independently of one another, H, alkyl, cycloalkyl, aryl or hetaryl or together with a further substituent of the compound of the formula (I) are part of a 4- to 8-membered ring system.

Particular preference is given to R¹ in the compounds of the formula (I) being —NH—C(═NH)NHR^{w}, where R^{w} is H, alkyl, cycloalkyl, aryl or hetaryl. R¹ is very particularly preferably —NH—C(═NH)NH₂.

R² and R³ in the compounds of the formula (I) are preferably in each case unsubstituted or substituted phenyl, pyridyl or cyclohexyl. R² and R³ are particularly preferably unsubstituted or substituted phenyl.

The indices a, b and c in the compounds of the formula (I) are preferably 0.

In a specific embodiment, the compounds of the formula (I) which are used according to the invention are selected among compounds of the formula (I.a),

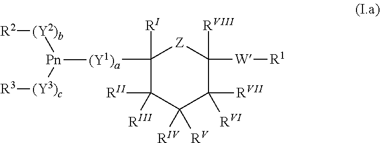

where
a, b, c, Pn, R¹, R², R³, Y′, Y² and Y³ each have one of the meanings given above,
W′ is a divalent bridging group having from 1 to 5 bridge atoms between the flanking bonds,
Z is O, S, S(═O), S(═O)₂, N(R^{IX}) or C(R^{X})(R^{X}) and
R^{I}, R^{II}, R^{III}, R^{IV}, R^{V}, R^{VI}, R^{VII}, R^{IX} and if present, R^{X} are each, independently of one another, H, halogen, nitro, cyano, amino, alkyl, alkoxy, alkylamino, dialkylamino, cycloalkyl, heterocycloalkyl, aryl or hetaryl,
   or two radicals R^{I}, R^{II}, R^{IV}, R^{VI}, R^{VIII} and R^{IX} bound to adjacent ring atoms together represent the second bond of a double bond between the adjacent ring atoms, with the six-membered ring being able to have up to three noncumulated double bonds.

As regards preferred meanings of a, b, c, Pn, R¹, R², R³, Y¹, Y² and Y³, reference is made to what has been said above with regard to the compounds of the general formula (I).

Compounds of the formula (I.a) in which a, b, c, Pn, R¹, R², R³, R^{I}, R^{II}, R^{III}, R^{IV}, R^{V}, R^{VI}, R^{VII}, R^{VIII}, R^{IX}, R^{X}, W′, Y¹, Y², Y³ and Z have, independently or preferably in combination, one of the meanings indicated above as preferred or one of the meanings mentioned below are particularly useful for the process of the invention.

W′ in the compounds of the formula (I.a) is preferably C₁-C₅-alkylene, (C₁-C₄-alkylene)carbonyl or C(═O). Particular preference is given to W′ in the compounds of the formula (I.a) being C(═O).

Z in the compounds of the formula (I.a) is preferably N(R^{IX}) or C(R^{IX})(R^{X}). Z is particularly preferably N(R^{IX}).

In the compound of the formula (I.a), preference is given to the radicals R^{I} together with R^{II}, R^{IV} together with R^{VI} and R^{VIII} together with R^{IX} in each case together representing the second bond of a double bond between the adjacent ring atoms, i.e. the six-membered ring in the compound of the formula (I.a) is preferably substituted benzene or pyridine.

Preference is given to the radicals R^{III}, R^{V}, R^{VII} and, if present, R^{X} in the compounds of the formula (I.a) each being, independently of one another, H, halogen, nitro, cyano, amino, C₁-C₄ alkyl, C₁-C₄-alkoxy, C₁-C₄-alkylamino or di(C₁-C₄-alkyl)amino. Particular preference is given to R^{III}, R^{V}, R^{VII} and, if present, R^{X} each being H.

In a particularly preferred embodiment of the process of the invention, the compounds of the formula (I) or (I.a) are selected from among the compounds of the formulae (I.1) and (I.2)

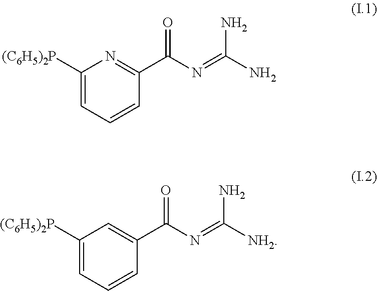

The compound of the formula (I.1) is very particularly preferably used for the hydroformylation in the process of the invention.

The catalysts used according to the invention have at least one compound of the formula (I) or (I.a). In addition to the above-described ligands, the catalysts can have at least one further ligand which is preferably selected from among halides, amines, carboxylates, acetylacetonate, arylsulfonates or alkylsulfonates, hydride, CO, olefins, dienes, cycloolefins, nitriles, N-comprising heterocycles, aromatics and heteroaromatics, ethers, PF₃, phospholes, phosphabenzenes and monodentate, bidentate and polydentate phosphine, phosphinite, phosphonite, phosphoramidite and phosphite ligands.

The catalysts used according to the invention comprise at least one metal of transition group VIII of the Periodic Table of the Elements. The metal of transition group VIII is preferably Co, Ru, Rh, Ir, Pd or Pt, particularly preferably Co, Ru, Rh or Ir and very particularly preferably Rh.

In general, catalytically active species of the general formula HₓM_{y}(CO)_{z}L_{q}, where M is the metal of transition group VIII, L is a pnicogen-comprising compound of the formula (I) and q, x, y, z are integers which depend on the valence and type of the metal and on the number of coordination sites occupied by the ligand L, are formed under hydroformylation conditions from the catalysts or catalyst precursors used in each case. Preference is given to z and q each being, independently of one another, at least 1, e.g. 1, 2 or 3. The sum of z and q is preferably from 1 to 5. The complexes can additionally comprise one or more of the above-described further ligands.

In a preferred embodiment, the hydroformylation catalysts are prepared in situ in the reactor used for the hydroformylation reaction. However, if desired, the catalysts according to the invention can also be prepared separately and be isolated by customary methods. To carry out the in-situ preparation of the catalysts according to the invention, it is possible, for example, to react at least one ligand of the formula (I) used according to the invention, a compound or a complex of a metal of transition group VIII, if appropriate at least one further additional ligand and if appropriate an activator in an inert solvent under the hydroformylation conditions.

Suitable rhodium compounds or complexes are, for example, rhodium(II) and rhodium(III) salts such as rhodium(III) chloride, rhodium(III) nitrate, rhodium(III) sulfate, potassium rhodium sulfate, rhodium(II) or rhodium(III) carboxylates, rhodium(II) and rhodium(III) acetate, rhodium(III) oxide, salts of rhodic(III) acid, trisammonium hexachlororhodate(III), etc.

Rhodium complexes such as dicarbonylrhodium acetylacetonate, acetylacetonatobisethylenerhodium(I), etc., are also suitable. Preference is given to using dicarbonylrhodium acetylacetonate or rhodium acetate.

Ruthenium salts or compounds are likewise suitable. Suitable ruthenium salts are, for example, ruthenium(III) chloride, ruthenium(IV), ruthenium(VI) or ruthenium(VIII) oxide, alkali metal salts of oxo acids of ruthenium, e.g. K₂RuO₄ or KRuO₄, or complexes such as RuHCl(CO)(PPh₃)₃. The carbonyls of ruthenium, e.g. dodecacarbonyltriruthenium or octadecacarbonylhexaruthenium, or mixed forms in which part of the CO has been replaced by ligands of the formula PR₃, e.g. Ru(CO)₃(PPh₃)₂, can also be used in the process of the invention.

Suitable cobalt compounds are, for example, cobalt(II) chloride, cobalt(II) sulfate, cobalt(II) carbonate, cobalt(II) nitrate, their amine or hydrate complexes, cobalt carboxylates such as cobalt acetate, cobalt ethylhexanoate, cobalt naphthanoate and the cobalt caproate complex. Here too, it is possible to use the carbonyl complexes of cobalt, e.g. octacarbonyldicobalt, dodecacarbonyltetracobalt and hexadecacarbonylhexacobalt.

The abovementioned and further suitable compounds of cobalt, rhodium, ruthenium and iridium are known in principle and are adequately described in the literature or they can be prepared by a person skilled in the art using methods analogous to those for the known compounds.

Suitable activators are, for example, Brønsted acids, Lewis acids such as BF₃, AlCl₃, ZnCl₂, and Lewis bases.

Suitable solvents are halogenated hydrocarbons such as dichloromethane or chloroform. Further suitable solvents are ethers such as tert-butyl methyl ether, diphenyl ether and tetrahydrofuran, esters of aliphatic carboxylic acids with alkanols, for example ethyl acetate or oxo oils such as Palatinol™ or Texanol™, aromatics such as toluene and xylenes, hydrocarbons or mixtures of hydrocarbons.

The molar ratio of monopnicogen ligand (I) to metal of transition group VIII is generally in the range from about 1:1 to 1000:1, preferably from 2:1 to 500:1 and particularly preferably from 5:1 to 100:1.

Preference is given to a process in which the catalyst is prepared in situ by reacting at least one ligand (II) as used according to the invention, a compound or a complex of a metal of transition group VIII and, if appropriate, an activator in an inert solvent under the hydroformylation conditions.

The decarboxylative hydroformylation reaction can be carried out continuously, semicontinuously or batchwise.

Suitable reactors for a continuous reaction are known to those skilled in the art and are described, for example, in Ullmanns Enzyklopädie der technischen Chemie, vol. 1, 3rd edition, 1951, p. 743 ff.

Suitable pressure-rated reactors are likewise known to those skilled in the art and are described, for example, in Ullmanns Enzyklopadie der technischen Chemie, vol. 1, 3rd edition, 1951, p. 769 ff. In general, the process of the invention is carried out using an autoclave which may, if desired, be provided with a stirrer and an internal liner.

The composition of the synthesis gas comprising carbon monoxide and hydrogen which is used in the process of the invention can vary within a wide range. The molar ratio of carbon monoxide and hydrogen is generally from about 5:95 to 70:30, preferably from about 40:60 to 60:40. Very particular preference is giving to using a molar ratio of carbon monoxide to hydrogen in the region of about 50:50.

The temperature in the hydroformylation reaction is generally in the range from about 10 to 180° C., preferably from about 20 to 120° C. In general, the pressure is in the range from about 1 to 700 bar, preferably from 1 to 400 bar, in particular from 1 to 200 bar. The reaction pressure can be varied as a function of the activity of the catalyst used. In general, the catalysts based on pnicogen-comprising compounds of the formula (I) which are used according to the invention allow the reaction to take place at low pressures, for instance in the range from 5 to 50 bar.

The catalysts used according to the invention can be separated off from the reaction product mixture by conventional methods known to those skilled in the art and can generally be reused as catalyst for the decarboxylative hydroformylation.

The above-described catalysts can also be immobilized in an appropriate way, e.g. by bonding via functional groups suitable as anchor groups, adsorption, grafting, etc., on a suitable support, e.g. glass, silica gel, synthetic resins, polymers, etc. They are then also suitable for use as solid-phase catalysts.

The catalysts used according to the invention advantageously display a high selectivity in respect of the carboxyl groups capable of forming intermolecular, noncovalent bonds in the α,β-unsaturated carboxylic acids used as substrates. Furthermore, the catalysts used according to the invention advantageously display a high activity, so that the corresponding aldehydes are generally obtained in high yields. In addition, isomerization of any further double bonds present does not occur or occurs only to a small extent when using the above-described catalysts.

Owing to the high selectivity of the catalysts used according to the invention in respect of the carboxyl group of the α,β-unsaturated carboxylic acid, high yields of the corresponding α,β-saturated aldehydes are obtained by the process of the invention regardless of the structure of the α,β-unsaturated carboxylic acid to be reacted.

The α,β-unsaturated carboxylic acids to be reacted by the process of the invention can have a number of functional groups, for example further nonconjugated C—C double bonds or C—C triple bonds or hydroxy, ether, acetal, amino, thioether, carbonyl, carboxyl, carboxylic ester, amido, carbamate, urethane, urea or silyl ether groups and/or substituents such as halogen, cyano or nitro. These functional groups and substituents do not undergo any reaction under the reaction conditions according to the invention.

In a specific embodiment of process of the invention, use is made of α,β-unsaturated carboxylic acids or salts thereof as can be obtained, for example, as natural or synthetic fatty acids or by industrial processes such as the oxo process, the SHOP (Shell higher olefin process) or Ziegler-Natta process or by metathesis. In this embodiment, the α,β-unsaturated carboxylic acids have predominantly linear alkyl or alkenyl radicals. These include, for example, straight-chain and branched C₈-C₃₂-alkyl, especially C₈-C₂₂-alkyl such as n-octyl, n-nonyl, n-decyl, n-undecyl, n-dodecyl, n-tridecyl, myristyl, pentadecyl, palmityl (═cetyl), heptadecyl, octadecyl, nonadecyl, arachinyl (arachidyl), behenyl, etc., and straight-chain and branched C₈-C₃₂-alkenyl, especially C₈-C₂₂-alkenyl, which may be monounsaturated or polyunsaturated, e.g. octenyl, nonenyl, decenyl, undecenyl, dodecenyl, tridecenyl, tetradecenyl, pentadecenyl, hexadecenyl, heptadecenyl, octadecenyl, nonadecenyl, linolyl, linolenyl, eleostearyl etc.

The process of the invention is particularly suitable for the decarboxylative hydroformylation of α,β-unsaturated carboxylic acids of the formula (II) or salts thereof,

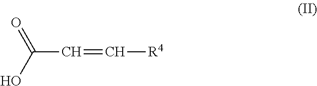

where
R⁴ is H, alkyl, alkenyl, alkynyl, cycloalkyl, heterocycloalkyl, aryl or hetaryl,
where one or more nonadjacent CH₂ groups in alkyl, alkenyl or alkynyl may independently be replaced by —O—, —O—C(═O)—, —O—Si(R^{4a})(R^{4b})—, —O—C(═O)—O—, —O—C(═O)—N(R^{4c})—, —O—C(═O)—S—, —N(R^{4c})—, —N(R^{4c}—C(═O)—, —N(R^{4c})—C(═O)—O—, —N(R^{4c})—C(═O)—N(R^{4c})—, —N(R^{4c})—C(═O)—S—, —S—, —S—C(═O)—, —S—C(═O)—O—, —S—C(═O)—N(R^{4c})—, —S—C(═O)—S—, —C(═O)—, —C(═O)—O—, —C(═O)—N(R^{c})—, —C(═O)—S— or —Si(R^{4a})(R^{4b})—, where
R^{4a} and R^{4b} are each, independently of one another, alkyl and
R^{4c} is H, alkyl, cycloalkyl, heterocycloalkyl, aryl or hetaryl,
where alkyl, alkenyl and alkynyl are unsubstituted or substituted by one or more substituents selected from among halogen, cyano, nitro, cycloalkyl, heterocycloalkyl, aryl and hetaryl and
where cycloalkyl, heterocycloalkyl, aryl and hetaryl are unsubstituted or substituted by 1 to 5 substituents selected from among alkyl and the substituents mentioned above for alkyl, alkenyl and alkynyl.

Starting from the compounds of the formula (III), the process of the invention gives aldehydes of the formula (III),

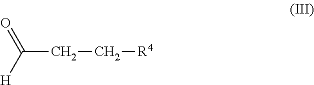

where R⁴ has the meaning given for the compound of the formula (II).

In the compounds of the formulae (II) and (III), R⁴ is especially H, alkyl, alkenyl, cycloalkyl or aryl, more especially alkyl or alkenyl and in particular C₁-C₂₀-alkyl or C₃-C₂₀-alkenyl, where one or more nonadjacent CH₂ groups in alkyl or alkenyl may independently be replaced as defined above and where alkyl, alkenyl, cycloalkyl and aryl are unsubstituted or substituted by one or more substituents.

If one or more nonadjacent CH₂ groups in alkyl, alkenyl or alkynyl have been replaced, the groups replacing CH₂ are preferably selected from among —O—, —O—C(═O)—, —O—Si(R^{4a})(R^{4b})—, —O—C(═O)—O—, —O—C(═O)—N(R^{4c})—, —N(R^{4c})—, —N(R^{4c})—C(═O)—, —N(R^{4c})—C(═O)—O—, N(R^{4c})—C(═O)—N(R^{4c})—, —S—, —S—C(═O)—, —C(═O)—, —C(═O)—O—, —C(═O)—N(R^{c})— and —C(═O)—S—. The groups replacing CH₂ are particularly preferably selected from among —O—, —O—C(═O)—, —O—Si(R^{4a})(R^{4b})—, —O—C(═O)—N(R^{4c})—, —N(R^{4c})—C(═O)—O—, —S—, —C(═O)— and —C(═O)—O—. In particular, if one or more nonadjacent CH₂ groups in alkyl, alkenyl or alkynyl have been replaced, from 1 to 5 and especially 1 or 2 CH₂ groups have been replaced by one of the abovementioned groups. In the abovementioned groups replacing CH₂, the radicals R^{4a} and R^{4b} are preferably selected independently from among C₁-C₂₀-alkyl and particularly preferably from among C₁-C₄-alkyl.

In the abovementioned groups replacing CH₂, the radicals R^{4c} are preferably selected independently from among H, alkyl, cycloalkyl and aryl, particularly preferably from among H, C₁-C₂₀-alkyl, C₅-C₈-cycloalkyl and phenyl, where alkyl, cycloalkyl and aryl are unsubstituted or substituted by 1 to 5 substituents.

If R⁴ in the compounds of the formulae (II) and (III) is alkyl, alkenyl or alkynyl, any substituents present are preferably selected independently from among cycloalkyl and aryl. Such substituents are particularly preferably selected independently from among C₃-C₇-cycloalkyl and phenyl. In particular, optionally substituted alkyl, alkenyl or alkynyl has from 1 to 5 substituents.

If R⁴ in the compounds of the formulae (II) and (III) is cycloalkyl, heterocycloalkyl, aryl or hetaryl, any substituents present are preferably selected independently from among alkyl, cycloalkyl and aryl. Such substituents are particularly preferably selected independently from among C₁-C₂₀-alkyl, especially C₁-C₁₂-alkyl, C₃-C₇-cycloalkyl and phenyl. In particular, optionally substituted cycloalkyl, heterocycloalkyl, aryl or hetaryl has from 1 to 5 substituents.

The invention further provides for the use of catalysts comprising at least one complex of a metal of transition group VIII with at least one ligand of the formula (I) as described above for the decarboxylative hydroformylation of α,β-unsaturated carboxylic acids. As regards preferred embodiments, reference is made to what has been said above with regard to the catalysts according to the invention.

The invention is illustrated below with the aid of nonlimiting examples.

### EXAMPLES

### I. General Conditions

The chemicals used were, unless indicated otherwise, commercially available. All reactions were carried out under a protective gas atmosphere (argon 5.0, Südwest-Gas) in dried glass apparatuses. Air- and moisture-sensitive liquids and solutions were transferred by means of a syringe. All solvents were dried and distilled using standard methods. Solutions were evaporated under reduced pressure on a rotary evaporator. Chromatographic purification was carried out using silica gel from Merck (Si 60®, 200-400 mesh). NMR spectra were recorded on a Varian Mercury spectrometer (300 MHz for ¹H-NMR; 75 MHz for ¹³C-NMR) or a Bruker AMX 400 (400 MHz for ¹H-NMR; 101 MHz for ¹³C-NMR) and were referenced by means of an internal TMS standard. ‘H-NMR data are reported as follows: chemical shift (δ in ppm), multiplicity (s=singlet; bs=broad singlet; d=doublet; t=triplet; q=quartet; m=multiplet), coupling constant (Hz), integration. ¹³C-NMR data are reported as chemical shift (δ in ppm). GC analysis was carried out using a 6890N AGILENT TECHNOLOGIES (column: 24079 SUPELCO, Supelcowax 10, 30.0 m×0.25 mm×0.25 μm; temperature: 175° C. isothermal; flow: He 1 ml/min; retention times: octanal (2.2 min), tetradecane (2.3 min), octanol (2.85 min.)). Elemental analysis were carried out on an Elementar vario (from Elementar Analysensystetne GmbH).

### II. General Preparative Methods

### Method A: General Method for the Preparation of α,β-Unsaturated Carboxylic Acids (by the Knoevenagel-Doebner Reaction)

An aldehyde (50.0 mmol, 1 eq.) is added at a temperature of 0° C. under an argon atmosphere to a solution of malonic acid (5.2 g, 50 mmol, 1 eq.) in a mixture of dry pyridine (8.09 ml, 100.0 mmol, 2 eq.) and pyrrolidine (41.4 μl, 0.5 mmol, 1 mol %, or 124 μl, 1.5 mmol, 3 mol % when branched aldehydes are reacted). The reaction mixture is stirred for 24 hours at room temperature (or 20 hours at room temperature and 4 hours at 60° C. when branched aldehydes are reacted). Phosphoric acid (20% strength, 60 ml) is added to the resulting reaction mixture at 0° C. The mixture is subsequently extracted with ethyl acetate (3×), dried over Na₂SO₄ and freed of the solvent under reduced pressure.

### Method B: General Method for the Decarboxylative Hydroformylation of α,β-Unsaturated Carboxylic Acids

The hydroformylation reactions were carried out in a stainless steel autoclave (Premex stainless steel autoclave Medintex, 100 ml) provided with a glass liner, magnetic stirrer (1000 rpm) and sample outlet. The hydroformylation solutions were prepared in a Schlenk flask [Rh(CO)₂acac], the compound of the formula I and if appropriate an internal standard (1,3,5-trimethoxybenzene for NMR analysis; tetradecane for GC analysis) and the solvent were placed in the flask. The α,β-unsaturated carboxylic acid was subsequently added to the mixture and the mixture was stirred under an argon atmosphere for 5 minutes. The reaction solution obtained was transferred under an argon atmosphere to the autoclave by means of a syringe. The autoclave was subsequently flushed three times with the synthesis gas (CO/H₂). The reaction was carried out under the conditions described below.

### III. Provision of the Compounds of the Formula (I)

Provision of N-(6-diphenylphosphanylpyridin-2-ylcarbonyl)guanidine (I.1)

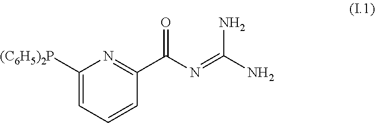

N-(6-Diphenylphosphanylpyridin-2-ylcarbonyl)guanidine (I.1) was prepared by the preparative method described in Angew. Chem. 2008, 120, 2, 317-321.

### IV. Kinetic Studies on the Reduction of Oct-2-Enoic Acid

For the kinetic studies, samples were taken from the reaction at the times indicated in tables 1 and 2 and examined by means of NMR analysis (after dilution with CDCl₃) or GC analysis (after filtration through a short silica gel column).

a) Decarboxylative Hydroformylation of Oct-2-Enoic Acid (10 Bar; CO/H₂, 1:1)

Oct-2-enoic acid was converted into the corresponding aldehyde using method B and the reaction conditions indicated below. The yields of the reaction products obtained based on the molar amount of the α,β-unsaturated carboxylic acid used are shown as a function of time in table 1.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of oct-2-enoic acid: c₀=0.2 M; solvent: CH₂Cl₂ (8 ml); pressure: 10 bar; synthesis gas: CO/H₂(1:1); reaction temperature: 25° C.

TABLE 1

Yield [%]

| Sample | Time [h] | Octanal | Octanol | Octanoic acid |
|---|---|---|---|---|
| 1 | 1.67 | 7.6 | 0 | n.d.*⁾ |
| 2 | 3.5 | 16 | 0 | n.d. |
| 3 | 6.0 | 26.1 | 0 | n.d. |
| 4 | 9.38 | 40.9 | 0 | n.d. |
| 5 | 21.17 | 86.6 | 0.2 | n.d. |
| 6 | 23.42 | 93.6 | 0.3 | <1 |
| 7 | 27.42 | 93.4 | 1 | <1 |

*⁾n.d. = not determined

b) Decarboxylative Hydroformylation of Oct-2-Enoic Acid (40 Bar; CO/H₂, 1:1)

Oct-2-enoic acid was converted into the corresponding aldehyde using method B and the reaction conditions indicated below. The yields of the reaction products obtained based on the molar amount of the α,β-unsaturated carboxylic acid used are shown as a function of time in table 2.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of oct-2-enoic acid: c₀=0.2 M; solvent: CH₂Cl₂ (8 ml); pressure: 40 bar; synthesis gas: CO/H₂, 1:7; reaction temperature: 25° C.

TABLE 2

Yield [%]

| Sample | Time [h] | Octanal | Octanol | Octanoic acid |
|---|---|---|---|---|
| 1 | 0.5 | 11.5 | 0 | n.d.*⁾ |
| 2 | 1.12 | 26 | 0 | n.d. |
| 3 | 2.37 | 49.8 | 0 | n.d. |
| 4 | 3.95 | 75 | 0.45 | n.d. |
| 5 | 5.3 | 90 | 1 | n.d. |
| 6 | 6.7 | 90.5 | 2 | n.d. |
| 7 | 20.53 | 80.3 | 10.5 | n.d. |
| 8 | 28.95 | 76.4 | 15.5 | n.d. |
| 9 | 48.0 | 67 | 23.5 | <1 |

*⁾n.d. = not determined

### VI. Preparative Examples

### Example 1

### Preparation of Octanal

### a) Provision of Trans-Oct-2-Enoic Acid

Hexanal was converted into trans-oct-2-enoic acid using method A. trans-Oct-2-enoic acid was isolated as a colorless liquid by Kugelrohr distillation (6.3 g, 89% yield). The product obtained comprised <1% of α,γ-isomer and 2% of trans-isomer. The NMR data obtained agreed with those for the commercially available product.

b) Decarboxylative hydroformylation of trans-oct-2-enoic acid

trans-Oct-2-enoic acid was converted into octanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of oct-2-enoic acid: c₀=0.2 M; solvent: CH₂Cl₂ (8 ml); pressure: 13 bar; synthesis gas: CO/H₂(1:1); reaction temperature: 25° C., reaction time: 24 h.

The yield of octanal prior to isolation was 94% according to GC analysis. Octanal was isolated in a yield of 75% (154 mg) from the crude product by bulb tube distillation. The NMR spectra obtained agreed with the literature.

### Example 2

### Preparation of Undecanal

trans-Undec-2-enoic acid was converted into undecanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of undec-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

Undecanal was isolated in a yield of 91% (248 mg) from the crude product by flash chromatography (petroleum ether/ethyl acetate, 10:1). The NMR spectra obtained agreed with the literature.

### Example 3

### Preparation of 3-Cyclohexylpropanal

a) Provision of Trans-3-Cyclohexylacrylic Acid

Cyclohexyl carboxaldehyde (30 mmol) was converted into trans-3-cyclohexylacrylic acid using method A. trans-3-Cyclohexylacrylic acid was isolated as a colorless crystalline solid (4.99 g, yield 78%) from the crude product by recrystallization from n-hexane (3 ml). The product obtained comprised <1% of β,γ-isomer and 1% of cis-isomer. The analytical data obtained agreed with the literature values.

b) Decarboxylative Hydroformylation of Trans-3-Cyclohexylacrylic Acid trans-3-Cyclohexylacrylic acid was converted into 3-cyclohexylpropanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-3-cyclohexylacrylic acid: c₀=0.2 M; solvent: CH₂Cl₂ (8 ml); pressure: 13 bar; synthesis gas: CO/H₂ (1:1); reaction temperature: 25° C., reaction time: 24 h.

According to the NMR spectrum of the crude product, the yield of 3-cyclohexylpropanal was 97%. 3-Cyclohexylpropanal was isolated in a yield of 166 mg (74%) from the crude product by bulb tube distillation. The NMR data of the product isolated agreed with the literature.

### Example 4

### Preparation of 4-methylpentanal

### a) Provision of Trans-4-Methylpent-2-Enoic Acid

2-Methylpropionic acid was converted into trans-4-methylpent-2-enoic acid using method A. trans-4-Methylpent-2-enoic acid was isolated as a colorless liquid (4.86 g, 85.1% yield) by bulb tube distillation. The product obtained comprised <1% of α,γ-isomer and <1% of cis-isomer. The NMR data agreed with those of the commercially available compound.

b) Decarboxylative Hydroformylation of Trans-4-Methylpent-2-Enoic Acid trans-Oct-2-enoic acid was converted into 4-methylpentanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-4-methylpent-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

According to the NMR spectrum of the crude product, the yield of 4-methylpentanal was 98%. 4-Methylpentanal was isolated as a volatile liquid (166 mg, 47% yield) by bulb tube distillation. The NMR data of the product isolated agreed with the literature.

### Example 5

### Preparation of 5-methylhexanal

### a) Provision Von Trans-5-Methylhex-2-Enoic Acid

3-Methylbutanoic acid was converted into trans-5-methylhex-2-enoic acid using method A. trans-5-Methylhex-2-enoic acid was isolated as a colorless liquid (6.1 g, yield 95.1%) by bulb tube distillation. The product obtained comprised 2.5% of α,γ-isomer and <1% of cis-isomer. The NMR data of the product isolated agreed with the literature.

### b) Decarboxylative Hydroformylation of Trans-5-Methylhex-2-Enoic Acid

trans-5-Methylhex-2-enoic acid was converted into 5-methylhexanal using method B and the reaction conditions indicated below.

Ligand: N-(6-diphenylphosphanylpyridin-2-ylcarbonyl)guanidine GO; molar ratio of: [Rh(CO)₂aeac]/(I.1)/carboxylic acid=1:10:200; starting concentration of trans-5-methylhex-2-enoic acid: c₀=0.2 M; solvent: CH₂Cl₂ (8 ml); pressure: 13 bar; synthesis gas: CO/H₂(1:1); reaction temperature: 25° C., reaction time: 24 h.

According to the NMR spectrum of the crude product, the yield of 5-methylhexanal was 97%. The NMR data of the product obtained agreed with the literature.

### Example 6

### Preparation of Dodec-6-Enal

### a) Provision of Trans,Trans-Dodeca-2,6-Dienoic Acid

trans-Dec-4-enal (20 mmol) comprising 9% of cis-isomer was converted into trans, trans-dodeca-2,6-dienoic acid using method A and the target compound was isolated from the crude product in an amount of 2.5 g (yield 63.7%) from the crude product by means of flash chromatography (petroleum ether/diethyl ether/acetic acid, 100:25:1). The product obtained comprised 1% of α,γ-isomer and 1.7% of 2-cis-isomer.

¹H-NMR (400.122 MHz, CDCl₃): δ=0.88 (t, J=7.1 Hz, 3H); 1.20-1.38 (m, 6H); 1.98 (_{pseudo}q;J=7.2 Hz); 2.16 (_{pseudo}q;J=6.9 Hz, 2H); 2.30 (_{pseudo}q, J=6.9 Hz, 2H); 5.29-5.50 (m, 2H); 5.83 (dt, J=15.6 HZ, J=1.6 Hz, 1H); 7.08 (dt, J=15.6 HZ, J=6.9 HZ, 1H), 11.8 ppm (vbs, 1H). ¹³C {¹H}-NMR (100.626 MHz, CDCl₃): δ=14.1; 22.6; 29.2; 30.9; 31.4; 32.4; 32.5; 121.0; 128.1; 132.1; 151.8; 172.3 ppm. Signals of the 6-cis-isomer (9%): ¹³C{¹H}-NMR (100.626 MHz; CDCl₃): δ=127.5; 131.6 ppm. MS-CI (chemical ionization, NH₃): (m/e)=214.2 (100%, [M+H+NH₃]⁺). Elemental analysis [% by weight]: calculated: C, 73.43; H, 10.17; found: C, 73.25; H, 9.94.

### b) Decarboxylative Hydroformylation of Trans,Trans-Dodeca-2,6-Dienoic Acid

trans,trans-Dodeca-2,6-dienoic acid was converted into trans-dodec-6-enal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans,trans-dodeca-2,6-dienoic acid: c₀=0.2 M; solvent: CH₂Cl₂ (8 ml); pressure: 13 bar; synthesis gas: CO/H₂ (1:1); reaction temperature: 25° C., reaction time: 24 h.

trans-Dodec-6-enal was isolated as a clear liquid (283 mg, yield 97%) from the reaction mixture by filtration through silica gel (washed 3 times with CH₂Cl₂) and subsequent removal of the solvent under reduced pressure. According to NMR analysis, the 6-(trans:cis) ratio was 91:9. This ratio corresponds to that of the starting compound.

¹H-NMR (400.132 MHz, CDCl₃): δ=0.88 (t, J=7.1 Hz, 3H); 1.20-1.43 (m, 8H); 1.64 (_{pseudo}q, J=7.6 Hz, 2H); 1.93-2.10 (m, 4H); 2.42 (dt, J=7.4 Hz, J=1.9 Hz, 2H); 5.32-5.45 (m, 2H); 9.76 ppm (t, J=1.8 Hz, 1H). ¹³C{¹}-NMR (100.626 MHz, CDCl₃): δ=14.2; 21.7; 22.7; 29.2; 29.4; 31.5; 32.4; 32.7; 43.9; 129.5; 131.3; 202.9 ppm. Signals of the 6-cis-isomer (9%): ¹³C {¹H}-NMR (100.626 MHz, CDCl₃): δ=129.0; 130.8 ppm. MS-CI (chemical ionization, NH₃): (m/e)=164.0 (28%); 200.1 (100%, [M+H+NH₃]⁺). Elemental analysis [% by weight]: calculated: C, 79.06; H, 12.16; found: 78.77; 11.96.

### Example 7

### Preparation of 5,9-Dimethyldec-8-Enal

a) Provision of trans-5,9-dimethyldeca-2,8-dienoic acid

3,7-Dimethyloct-6-enal was converted into trans-5,9-dimethyldeca-2,8-dienoic acid using method A. trans-5,9-Dimethyldeca-2,8-dienoic acid was isolated as a colorless liquid (8.32 g, 85% yield) by distillation under reduced pressure. The product obtained comprised 3% of f β,γ-isomer and <1% of cis-isomer.

¹H-NMR (400.132 MHz, CDCl₃): δ=0.92 (d, J=6.7 Hz, 3H); 1.15-1.41 (m, 2H); 1.58-1.71 (m, 1H); 1.60 (d, J=0.9 Hz, 3H); 1.68 (d, J=1.1 Hz. 3H); 1.91-2.29 (m, 4H); 5.08 (tm, J=7.1 Hz, 1H); 5.83 (dt, J=15.6 Hz, J=1.5 Hz, 1H); 7.07 (dt, J=15.6 Hz, J=7.6 Hz, 1H); 11.9 ppm (vbs, 1H). ¹³C{¹}-NMR (100.626 MHz, CDCl₃): δ=17.7; 19.5; 25.5; 25.7; 32.1; 36.7; 39.7; 121.8; 124.3; 131.5; 151.3; 172.2 ppm. MS-CI (chemical ionization, NH₃): (m/e)=197.1 (6%; [M+H]⁺); 214.2 (100%, [M+H+NH₃]⁺). Elemental analysis [% by weight]: calculated: C, 73.43; H, 10.27; found: C, 73.21; H, 10.24.

b) Decarboxylative hydroformylation of trans-5,9-dimethyldeca-2,8-dienoic acid trans-5,9-Dimethyldeca-2,8-dienoic acid was converted into 5,9-dimethyldec-8-enal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-5,9-dimethyldeca-2,8-dienoic acid: c₀=0.2 M; solvent: CH₂Cl₂ (8 ml); pressure: 13 bar; synthesis gas: CO/H₂ (1:1); reaction temperature: 25° C., reaction time: 24 h.

5,9-Dimethyldec-8-enal was isolated as a clear liquid (274 mg, 94% yield) by filtration of the reaction mixture through silica gel (washed 3 times with CH₂Cl₂) and subsequent removal of the solvent under reduced pressure.

‘H-NMR (400.132 MHz, CDCl₃): δ=0.89 (d, J=6.6 Hz, 3H); 1.1-1.73 (m, 7H); 1.60 (d, J=0.8 Hz, 3H); 1.68 (d, J=1.3 Hz, 3H); 1.88-2.04 (m, 2H); 2.40 (dt, J=7.6 Hz, J=1.9 Hz, 2H); 5.09 (tm, J=7.1 Hz, 1H); 9.76 ppm (t, J=1.8 Hz, 1H). ¹³C {¹H}-NMR (100.626 MHz, CDCl₃): δ=17.7; 19.5; 19.7; 25.6; 25.8; 32.3; 36.5; 37.0; 44.3; 124.9; 131.3; 202.9 ppm. MS-CI (chemical ionization, NH₃): (m/e)=200.1 (100% [M+H+NH₃]⁺). Elemental analysis [% by weight]: calculated: C, 79.06; H, 12.16; found: C, 78.71; H, 11.92.

### Example 8

### Preparation of 7-Phenylheptanal

trans-7-Phenylhept-2-enoic acid was converted into 7-phenylheptanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-7-phenylhept-2-enoic acid: c₀=0.2 M; solvent: CH₂Cl₂ (8 ml); pressure: 13 bar; synthesis gas: CO/H₂(1:1); reaction temperature: 25° C., reaction time: 24 h.

7-Phenylheptanal was isolated as a clear liquid (286 mg, 94% yield) by filtration of the reaction mixture through silica gel (washed 3 times with CH₂Cl₂) and subsequent removal of the solvent under reduced pressure. The NMR data of the product isolated agreed with the literature.

### Example 9

### Preparation of 12-Hydroxydodecanal

trans-12-Hydroxydodec-2-enoic acid was converted into 12-hydroxydodecanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-2-hydroxydodec-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

12-Hydroxydodecanal was isolated as a colorless solid (279 mg, yield 87%) by filtration of the reaction mixture through silica gel (washed 3 times with CH₂Cl₂/diethyl ether (2:1)) and subsequent removal of the solvent under reduced pressure. The NMR data of the product isolated agreed with the literature.

### Example 10

### Preparation of 8-Oxononanal

trans-8-Oxonon-2-enoic acid was converted into 8-oxononanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/oct-2-enoic acid=1:10:200; starting concentration of trans-8-oxonon-2-enoic acid: c₀=0.2 M; solvent: CH₂Cl₂ (8 ml); pressure: 13 bar; synthesis gas: CO/H₂(1:1); reaction temperature: 25° C., reaction time: 24 h.

8-Oxononanal was isolated as a clear liquid (227 mg, 91% yield) by filtration of the reaction mixture through silica gel (washed twice with CH₂Cl₂/diethyl ether (10:1)) and subsequent removal of the solvent under reduced pressure. The NMR data of the product isolated agreed with the literature.

### Example 11

### Preparation of 5-methylsulfanylpentanal

### a) Provision of Trans-5-Methylsulfanylpent-2-Enoic Acid

3-Methylsulfanylpropanal was converted into trans-5-methylsulfanylpent-2-enoic acid using method A. The crude product obtained (6.97 g) comprised 18% of the β,γ-isomer. Purification by column chromatography (petroleum ether/diethyl ether/acetic acid, 100:50:1) gave 3.32 g of trans-5-methylsulfanylpent-2-enoic acid (yield 45%). The product isolated comprised 5.7% of the β,γ-isomer and <1% of the cis-isomer.

¹H-NMR (400.132 MHz, CDCl₃): δ=1.51-1.57 (m, 2H); 2.13 (s, 3H); 2.62-2.66 (m, 2H); 5.89 (dt, J=15.7 Hz, J=1.5 Hz, 1H); 7.09 (dt, J=15.7 Hz, J=6.8 Hz, 1H); 11.9 ppm (vbs, 1H). ¹³C{¹1H}-NMR (100.626 MHz, CDCl₃): δ=15.6; 31.9; 32.4; 121.9; 171.9 ppm.

MS-CI (chemical ionization, NH₃): (m/e)=163.9 (100%, [M+H+NH₃]⁺). Elemental analysis [% by weight]: calculated: C, 49.29; H, 6.89; found: C, 48.90; H, 6.82.

### b) Decarboxylative Hydroformylation of Trans-5-Methylsulfanylpent-2-Enoic Acid

trans-5-Methylsulfanylpent-2-enoic acid was converted into 5-methylsulfanylpentanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-5-methylsulfanylpent-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

5-Methylsulfanylpentanal was isolated as a clear liquid (165 mg, 78% yield) by filtration of the reaction mixture through silica gel (washed twice with CH₂Cl₂) and subsequent removal of the solvent under reduced pressure. The sample examined comprised 4% of 5-methylsulfanylpentan-1-ol. The NMR data of the product isolated agreed with the literature.

### Example 12

### Preparation Of 9-Oxononyl Benzoate

trans-9-Benzoyloxynon-2-enoic acid was converted into 9-oxononyl benzoate using method B and the following reaction conditions.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-9-benzoyloxynon-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

9-Oxononyl benzoate was isolated as a clear liquid (403 mg, yield 96%) by filtration of the reaction mixture through silica gel (washed twice with CH₂Cl₂) and subsequent removal of the solvent under reduced pressure. The NMR data of the product isolated agreed with the literature.

### Example 13

### Preparation of 9-benzyloxynonanal

trans-9-Benzyloxynon-2-enoic acid was converted into 9-benzyloxynonanal using method B and the reaction conditions indicated below.

Ligand: N-(6-diphenylphosphanylpyridin-2-ylcarbonyl)guanidine (IA); molar ratio of: [Rh(CO)₂acac]/(I.1)/carboxylic acid=1:10:200; starting concentration of trans-9-benzyloxynon-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

9-Benzyloxynonanal was isolated in a yield of 298 mg (75%) from the crude product by flash chromatography (cyclohexane/diethyl ether, 6:1). The NMR data of the product isolated agreed with the literature.

### Example 14

### Preparation of 9-(Tert-Butyldimethylsilanyloxy)Nonanal

trans-9-(tert-Butyldimethylsilanyloxy)non-2-enoic acid was converted into octanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-9-(tert-butyldimethylsilanyloxy)non-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

9-(tent-Butyldimethylsilanyloxy)nonanal was isolated as a clear liquid (415 mg, 95% yield) by filtration of the reaction mixture through silica gel (washed twice with CH₂Cl₂) and subsequent removal of the solvent under reduced pressure. The NMR data of the product isolated agreed with the literature.

### Example 15

### Preparation of 14,14-Dimethoxytetradecanal

trans-14,14-Dimethoxytetradec-2-enoic acid was converted into 14,14-dimethoxytetradecanal using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of trans-14,14-dimethoxytetradec-2-enoic acid: c₀=0.2 M; solvent: CH₂Cl₂ (8 ml); pressure: 13 bar; synthesis gas: CO/H₂ (1:1); reaction temperature: 25° C., reaction time: 24 h.

14,14-Dimethoxytetradecanal was isolated in a yield of 292 mg (67%) by flash chromatography (cyclohexane/diethyl ether, 6:1).

¹H-NMR (400.132 MHz, CDCl₃): δ=1.21-1.37 (m, 18H); 1.56-1.66 (m, 4H); 2.42 (dt, J=7.4 Hz, J=1.9 Hz, 2H); 3.31 (s, 6H); 4.35 (t, J=5.7 Hz, 1H); 9.76 ppm (t, J=1.9 Hz, 1H). ¹³C {¹H)—NMR (100.626 MHz, CDCl₃): δ=22.2; 24.7; 29.2; 29.41: 29.48; 29.55; 29.60: 29.61; 29.65; 32.58; 44.0; 52.7; 104.7; 203.0 ppm. MS-CI (chemical ionization, NH₃): (m/e)=226.1 (57%); 241.1 (100%, [M+H—CH₃OH]⁺); 258.2 (42%, [M+H+NH₃—C₃OH]⁺); 290.2 (4%, [M+H+NH₃]⁺). Elemental analysis [% by weight]: calculated: C, 70.54; H, 11.94; found: C, 70.69; H, 11.70.

### Example 16

### Preparation of 9-Oxononyl N-Phenylcarbamate

8-Hydroxycarbonyloct-7-enyl trans-N-phenylcarbamate was converted into 9-oxononyl N-phenylcarbamate using method B and the reaction conditions indicated below.

Ligand: N-(6-diphenylphosphanylpyridin-2-ylcarbonyl)guanidine GO; molar ratio of: [Rh]/(I.1)/carboxylic acid=1:10:100; starting concentration of 8-hydroxycarbonyloct-7-enyl trans-N-phenylcarbamate: c₀=0.1 M; solvent: CH₂Cl₂ (8 ml); pressure: 20 bar; synthesis gas: CO/H₂(1:1); reaction temperature: 25° C., reaction time: 20 h.

9-Oxononyl N-phenylcarbamate was isolated as a white solid (341.7 mg, yield 77%) from the crude product by flash chromatography (cyclohexane/ethyl acetate, 3:1).

¹H-NMR (400.132 MHz, CDCl₃): δ=1.29-1.43 (m, 8H); 1.59-1.71 (m, 4H); 2.42 (dt, J=7.3 Hz, J=1.8 Hz, 2H); 4.15 (t, J=6.6 Hz, 2H); 6.68 (bs, 1H); 7.05 (tt, J=7.3 Hz, J=1.2 Hz, 1H); 7.26-7.32 (m, 2H); 7.38 (bd, J=7.8 Hz, 2H); 9.76 ppm (t, J=1.8 Hz, 1H). ¹³C{¹H}-NMR (100.626 MHz, CDCl₃): δ=22.1; 25.8; 28.9; 29.0; 29.1; 29.2; 43.9; 65.4; 118.7; 123.4; 129.1; 138.1; 153.8; 202.9 ppm. MS-CI (chemical ionization, NH₃): (m/e)=278.2 (100%, [M+H]⁺). Elemental analysis [% by weight]: calculated: C, 69.29; H, 8.36; found: C, 69.18; H, 8.41.

### Example 17

### Preparation of 12-Oxododecanoic Acid

### a) Provision of Trans-Dodec-2-Enedicarboxylic Acid

10-Oxodecanoic acid was converted into trans-dodec-2-enedicarboxylic acid using method A and 4 molar equivalents of pyridine. The crude product was recrystallized from ethyl acetate.

trans-Dodec-2-enedicarboxylic acid was isolated as a colorless solid (903 mg; yield 79%; <1% of β,γ-isomer; <1% of cis-isomer).

¹H-NMR (400.132 MHz, DMSO-d⁶): δ=1.20-1.30 (m, 8H); 1.35-1.44 (m, 2H); 1.44-1.53 (m, 2H); 2.12-2.21 (m, 4H); 5.75 (dt, J=15.6 Hz, J=1.5 Hz, 1H), 6.81 (dt, J=15.6 Hz, J=7.0 Hz, 1H), 11.5 ppm (vbs, 1H). ¹³C{¹H}-NMR (100.626 MHz, DMSO-d⁶): δ=24.4; 27.4; 28.5; 28.6; 31.3; 33.6; 121.8; 148.8; 167.0; 174.4 ppm. MS-CI (chemical ionization, NH₃): (m/e)=229.2 (35%, [M+H]⁺); 246.2 (100%, [M-1-H+NH₃]⁺). Elemental analysis [% by weight]: calculated: C, 63.14; H, 8.83; found: 62.79H, 8.88.

### b) Decarboxylative Hydroformylation of Trans-Dodec-2-Enedicarboxylic Acid

trans-Dodec-2-enedicarboxylic acid was converted into 12-oxododecananoic acid using method B and the reaction conditions indicated below.

Ligand: N-(6-diphenylphosphanylpyridin-2-ylcarbonyl)guanidine (I.1); molar ratio of: [Rh(CO)₂acac]/(I.1)/carboxylic acid=1:10:200; starting concentration of 12-oxododecanoic acid: c₀=0.2 M; solvent: CH₂Cl₂ (8 ml); pressure: 13 bar; synthesis gas: CO/H₂ (1:1); reaction temperature: 25° C., reaction time: 24 h.

12-Oxododecanoic acid was isolated as a colorless solid (171.5 mg, yield 50%) from the crude product by flash chromatography (petroleum ether/diethyl ether/acetic acid=100:50:1). 75% of the starting compound had reacted. The analytical data obtained agree with the literature.

### VII. Molecular Modeling

The capability of mutual recognition of catalyst and α,β-unsaturated carboxylic acids was checked by molecular modeling (MMFF, Spartan Pro). The data obtained support the experimental findings with regard to the mutual recognition of catalyst and substrate.

1.-19. (canceled)

20. A process for preparing an aldehyde which comprises reacting an α,β-unsaturated carboxylic acid or a salt thereof with carbon monoxide and hydrogen in the presence of a catalyst comprising at least one complex of a metal of transition group VIII of the Periodic Table of the Elements with at least one compound of the formula (I), 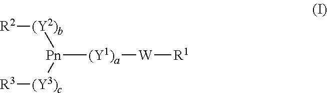
wherein
Pn is a pnicogen atom;
W is a divalent bridging group having from 1 to 8 bridge atoms between the flanking bonds;
R¹ is a functional group capable of forming intermolecular, noncovalent bonds with the carboxyl group of the α,β-unsaturated carboxylic acid;
R² and R³ are each, independently of one another, alkyl, cycloalkyl, heterocycloalkyl, aryl or hetaryl, where alkyl is unsubstituted or substituted by 1, 2, 3, 4 or 5 substituents selected from halogen, cyano, nitro, alkoxy, cycloalkyl, cycloalkoxy, heterocycloalkyl, heterocycloalkoxy, aryl, aryloxy, hetaryl or hetaryloxy and where cycloalkyl, heterocycloalkyl, aryl and hetaryl are unsubstituted or substituted by 1, 2, 3, 4 or 5 substituents selected from alkyl or the substituents mentioned above for alkyl; or
together with the pnicogen atom and together with the groups Y² and Y³ if present form a 5- to 8-membered heterocycle which may additionally be fused with one, two, three or four cycloalkyl, heterocycloalkyl, aryl or hetaryl groups, where the heterocycle and, if present, the fused-on groups each have, independently of one another, 1, 2, 3, 4 or 5 substituents selected from halogen, cyano, nitro, alkyl, alkoxy, cycloalkyl, cycloalkoxy, heterocycloalkyl, heterocycloalkoxy, aryl, aryloxy, hetaryl or hetaryloxy,
a, b and c are each, independently of one another, 0 or 1 and
Y¹, Y² and Y³ are each, independently of one another, O, S, NR^{a} or SiR^{b}R^{c}, where R^{a}, R^{b} and R^{c} are each, independently of one another, hydrogen, alkyl, cycloalkyl, heterocycloalkyl, aryl or hetaryl, where alkyl is unsubstituted or substituted by 1, 2, 3, 4 or 5 substituents selected from halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl are unsubstituted or substituted by 1, 2, 3, 4 or 5 substituents selected from alkyl or the substituents mentioned above for alkyl.

21. The process according to claim 20, wherein the number of carbon atoms of the aldehyde produced corresponds to the number of carbon atoms of the α,β-unsaturated carboxylic acid used.

22. The process according to claim 20, wherein the catalyst is capable of forming, by means of the group R¹, an aggregate with the α,β-unsaturated carboxylic acid, with the conjugated C—C double bond of the α,β-unsaturated carboxylic acid being capable of interacting with the complexed metal of transition group VIII.

23. The process according to claim 20, wherein the metal of transition group VIII of the Periodic Table of the Elements is selected from Co, Ru, Rh, Ir, Pd or Pt.

24. The process according to claim 23, wherein the metal of transition group VIII of the Periodic Table is Rh.

25. The process according to claim 20, wherein Pn in the compounds of the formula (I) is phosphorus.

26. The process according to claim 20, wherein the radical R¹ in the compound of the formula (I) comprises at least one NH group.

27. The process according to claim 26, wherein R¹ is selected from —NHR^{w}, ═NH, —C(═O)NHR^{w}, —C(═S)NHR^{w}, —C(═NR^{y})NHR^{w}, —O—C(═O)NHR^{w}, —O—C(═S)NHR^{w}, —O—C(═NR^{y})NHR^{w}, —N(R^{z})—C(═O)NHR^{w}, —N(R^{z})—C(═S)NHR^{w} or —N(R^{z})—C(═NR^{y})NHR^{w}, where R^{w}, R^{y} and R^{z} are each, independently of one another, H, alkyl, cycloalkyl, aryl or hetaryl or together with a further substituent of the compound of the formula (II) are part of a 4- to 8-membered ring system.

28. The process according to claim 26, wherein R¹ is —NH—C(═NH)NHR^{w}, where R^{w} is H, alkyl, cycloalkyl, aryl or hetaryl.

29. The process according to claim 20, wherein R² and R³ are selected from in each case optionally substituted phenyl, pyridyl or cyclohexyl.

30. The process according to claim 20, wherein a, b and c are 0.

31. The process according to claim 20, wherein the compound of the formula (I) is selected from compounds of the formula (I.a), 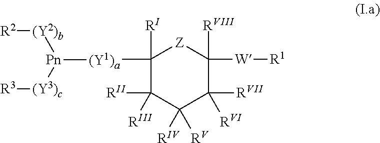
wherein
W′ is a divalent bridging group having from 1 to 5 bridge atoms between the flanking bonds,
Z is O, S, S(═O), S(═O₂), N(R^{IX}) or C(R^{IX})(R^{X}) and
R^{I}, R^{II}, R^{III}, R^{IV}, R^{V}, R^{VI}, R^{VII}, R^{VIII}, R^{IX}, and R^{X} are each, independently of one another, H, halogen, nitro, cyano, amino, alkyl, alkoxy, alkylamino, dialkylamino, cycloalkyl, heterocycloalkyl, aryl or hetaryl,
or two radicals R^{I}, R^{II}, R^{IV}, R^{VI}, R^{VIII} and R^{IX} bound to adjacent ring atoms together represent the second bond of a double bond between the adjacent ring atoms, with the six-membered ring being able to have up to three noncumulated double bonds.

32. The process according to claim 31, wherein W in the compound of the formula (I.a) is C(═O).

33. The process according to claim 31, wherein R¹ in the compounds of the formula (I.a) is —NH—C(═NH)NHR^{w}, where R^{W} is H, alkyl, cycloalkyl, aryl or hetaryl.

34. The process according to claim 31, wherein the radicals R^{I} together with R^{II}, R^{IV} together with R^{VI} and R^{VIII} together with R^{IX} in the compound of the formula (I.a) in each case together represent the second bond of a double bond between the adjacent ring atoms.

35. The process according to claim 20, wherein an α,β-unsaturated carboxylic acid of the formula (II), 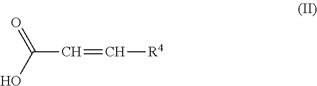
where
R⁴ is H, alkyl, alkenyl, alkynyl, cycloalkyl, heterocycloalkyl, aryl or hetaryl,
where one or more nonadjacent CH₂ groups in alkyl, alkenyl or alkynyl is optionally independently replaced by —O—, —O—C(═O)—, —O—Si(R^{4a})(R^{4b})—, —O—C(═O)—O—, —O—C(═O)—N(R^{4c})—, —O—C(═O)—S—, —N(R^{4c})—, —N(R^{4c})—C(═O)—, —N(R^{4c})—C(═O)—O—, —N(R^{4c})—C(═O)—N(R^{4c})—, —N(R^{4c})—C(═O)—S—, —S—, —S—C(═O)—, —S—C(═O)—O—, —S—C(═O)—N(R^{4c})—, —S—C(═O)—S—, —C(═O)—, —C(═O)—O—, —C(═O)—N(R^{c})—, —C(═O)—S— or —Si(R^{4a})(R^{4b})—, where
R^{4a} and R^{4b} are each, independently of one another, alkyl and
R^{4c} is H, alkyl, cycloalkyl, heterocycloalkyl, aryl or hetaryl,
where alkyl, alkenyl and alkynyl are unsubstituted or substituted by one or more substituents selected from among halogen, cyano, nitro, cycloalkyl, heterocycloalkyl, aryl and hetaryl and
where cycloalkyl, heterocycloalkyl, aryl and hetaryl are unsubstituted or substituted by one or more substituents selected from among alkyl and the substituents mentioned above for alkyl, alkenyl and alkynyl,
is converted into an aldehyde of the formula (III), 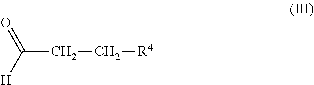
where R⁴ has the meanings given for the compound of the formula (II).

36. A process for the decarboxylative hydroformylation of α,β-unsaturated carboxylic acids which comprises utilizing a catalyst comprising at least one complex of a metal of transition group VIII of the Periodic Table of the Elements with at least one compound of the formula (I), 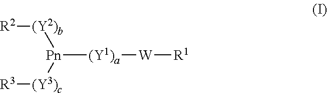
where
Pn is a pnicogen atom;
W is a divalent bridging group having from 1 to 8 bridge atoms between the flanking bonds;
R¹ is a functional group capable of forming intermolecular, noncovalent bonds with the carboxyl group of the α,β-unsaturated carboxylic acid;
R² and R³ are each, independently of one another, alkyl, cycloalkyl, heterocycloalkyl, aryl or hetaryl, where alkyl is unsubstituted or substituted by 1, 2, 3, 4 or 5 substituents selected from halogen, cyano, nitro, alkoxy, cycloalkyl, cycloalkoxy, heterocycloalkyl, heterocycloalkoxy, aryl, aryloxy, hetaryl or hetaryloxy and where cycloalkyl, heterocycloalkyl, aryl and hetaryl are unsubstituted or substituted by 1, 2, 3, 4 or 5 substituents selected from alkyl or the substituents mentioned above for alkyl; or
together with the pnicogen atom and together with the groups Y² and Y³ if present form a 5- to 8-membered heterocycle which may additionally be fused with one, two, three or four cycloalkyl, heterocycloalkyl, aryl or hetaryl groups, where the heterocycle and, if present, the fused-on groups each have, independently of one another, 1, 2, 3, 4 or 5 substituents selected from halogen, cyano, nitro, alkyl, alkoxy, cycloalkyl, cycloalkoxy, heterocycloalkyl, heterocycloalkoxy, aryl, aryloxy, hetaryl or hetaryloxy,
a, b and c are each, independently of one another, 0 or 1 and
Y¹, Y² and Y³ are each, independently of one another, O, S, NR^{a} or SiR^{b}R^{c}, where R^{a}, R^{b} and R^{c} are each, independently of one another, hydrogen, alkyl, cycloalkyl, heterocycloalkyl, aryl or hetaryl, where alkyl is unsubstituted or substituted by 1, 2, 3, 4 or 5 substituents selected from halogen, cyano, nitro, alkoxy, cycloalkyl, cycloalkoxy, heterocycloalkyl, heterocycloalkoxy, aryl, aryloxy, hetaryl and hetaryloxy and where cycloalkyl, heterocycloalkyl, aryl and hetaryl are unsubstituted or substituted by 1, 2, 3, 4 or 5 substituents selected from alkyl or the substituents mentioned above for alkyl.

37. The process according to claim 36, wherein the metal of transition group VIII of the Periodic Table of the Elements is selected from among Co, Ru, Rh, Ir, Pd and Pt.

38. The process according to claim 36, wherein the compound of the formula (I) is selected from among compounds of the formula (I.a) 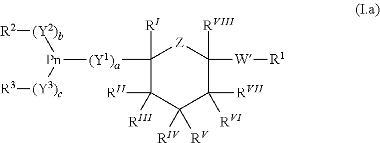
wherein
W′ is a divalent bridging group having from 1 to 5 bridge atoms between the flanking bonds,
Z is O, S, S(═O), S(═O₂), N(R^{IX}) or C(R^{IX})(R^{X}) and
R^{I}, R^{II}, R^{III}, R^{IV}, R^{V}, R^{VI}, R^{VII}, R^{VIII}, R^{IX} and R^{X} are each, independently of one another, H, halogen, nitro, cyano, amino, alkyl, alkoxy, alkylamino, dialkylamino, cycloalkyl, heterocycloalkyl, aryl or hetaryl,
or two radicals R^{I}, R^{II}, R^{IV}, R^{VI}, R^{VIII} and R^{IX} bound to adjacent ring atoms together represent the second bond of a double bond between the adjacent ring atoms, with the six-membered ring being able to have up to three noncumulated double bonds.
